(19) United States
(12) Patent Application Publication
(10) Pub. No.: US 2008/0139624 A1
(43) Pub. Date: Jun. 12, 2008

(54) Oral Dosage Form Comprising Rosiglitazone

(76) Inventor: Vincenzo Re, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/815,333

(22) PCT Filed: Feb. 3, 2006
(86) PCT No.: PCT/EP06/00999

§ 371 Date: Aug. 2, 2007

(30) Foreign Application Priority Data
Feb. 7, 2005 (GB) 0502479.9

(51) Int. Cl. A61K 31/4439; A61P 25/28; A61P 3/10; A61P 11/06

(52) U.S. Cl. 514/342

(57) ABSTRACT

An oral dosage form comprising pellets of a first composition and pellets of a second composition, each composition comprising 5-[4-[2-(N-methyl-N-(2 pyridyl)amino)ethoxy]benzyl]thiazolidine-2,4-dione or a pharmaceutically acceptable salt or solvate thereof and a pharmaceutically acceptable carrier therefor, wherein the first and second compositions are arranged to release drug at differing release rates on administration, preferably such that the rate of release of the drug from the dosage form is substantially independent of pH; a process for preparing such a dosage form and the use of such a dosage form in medicine.

The present invention relates to an oral dosage form comprising 5-[4-[2-(N-methyl-N-(2 pyridyl)amino)ethoxy]benzyl]thiazolidine-2,4-dione (hereinafter ‘Compound A’) or a pharmaceutically acceptable salt or solvate thereof, to a process for preparing such a dosage form and to the use of such a dosage form in medicine.

The use of a coating to control the rate of release of an active agent has received considerable attention and many different devices have been developed for such a purpose. For example, International Patent Application, Publication Number WO 01/05430 describes a drug delivery device that enables the delivery of drug substances which exhibit pH dependent solubility, in particular compounds that are more soluble at low pH levels (less than pH 2) than at near neutral pH levels (greater than about pH 5). Such delivery devices are characterised by the presence of a coating that is impermeable and insoluble in the fluid of the environment of use.

International patent application, Publication Number WO 95/30422 describes a series of controlled-release dosage forms of azithromycin. In particular, there is described a series of dosage forms that reduce the exposure of the upper GI tract (e.g. the stomach) to high concentrations of azithromycin, by the use of a pH dependent coating. Such dosage forms do not feature openings through which release of the drug substance may occur.

U.S. Pat. No. 6,099,859 describes a controlled release tablet for the delivery of an antihyperglycaemic drug, which comprises an osmotically active drug-containing core and a semipermeable membrane, wherein the semipermeable membrane is permeable to the passage of water and biological fluids and is impermeable to the passage of the drug substance. The semipermeable membrane contains at least one passageway for the release of the antihyperglycaemic drug.

U.S. Pat. No. 5,543,155 describes a diffusion-osmotic controlled drug release pharmaceutical composition comprising a one- or two-layer tablet core containing hydroxypropyl methylcellulose, said core having a film-coat comprising an ammonium methacrylate copolymer.

Additional devices that utilise a coating to control the rate of release of an active agent are discussed in U.S. Pat. No. 5,004,614. This patent describes a tablet core provided with an outer coating that is substantially impermeable to environmental fluid. The said outer coating may be prepared from materials that are either insoluble or soluble in the environmental fluids. Where a soluble material is used, the coating is of sufficient thickness that the core is not exposed to environmental fluid before the desired duration of the controlled release of the active agent has passed. Through this impermeable outer coating, one or more opening(s) has been created, so as to provide environmental fluids with an access route to the core. Therefore, upon ingestion of the coated tablet, gastrointestinal fluid can enter the opening(s) and contact or penetrate the core, to release the active agent. The result is that the active agent is released in a controlled manner out of the opening(s) only. The preferred geometry is such that there is a circular hole on the top and bottom face of the coated tablet. The opening(s) in question have an area from about 10 to 60 percent of the face area of the coated tablet. The rate of drug release is found to be directly related to the diameter of the opening(s) and to the solubility of the matrix core and active agent, allowing the possibility for a variety of drug release profiles be it zero or first order release.

The substantially impermeable coatings of U.S. Pat. No. 5,004,614 are not suitable for the controlled release of all active agents, especially pharmaceutically active weak bases or pharmaceutically acceptable salts and solvates thereof. Such active agents exhibit a marked pH dependent solubility, i.e. they are more soluble at around pH 2, associated with regions found in the stomach, compared to their solubility in the generally neutral conditions of the small intestine, around pH 7.

International Patent Application, Publication Number WO 03/068195 discloses an oral dosage form comprising an erodable core which contains a pharmaceutically active weak base or a pharmaceutically acceptable salt or solvate thereof, such as Compound A, the core having a coating with one or more openings leading to the core, and the coating being erodable under predetermined pH conditions. This provides a beneficial means for administration of a pharmaceutically active weak base or a pharmaceutically acceptable salt or solvate thereof, such as Compound A, where it is desirable that release of the active compound takes place in more than one pH environment, based on the finding that it is also beneficial for the coating to be erodable or soluble in a pH dependent manner.

European Patent Application, Publication Number 0 306 228 A1 relates to certain thiazolidinedione derivatives disclosed as having antihyperglycaemic and hypolipidaemic activity. One particular thiazolidinedione disclosed in EP 0 306 228 A1 is Compound A. International Patent Application, Publication Number WO 94/05659 discloses certain salts of Compound A including the maleate salt at Example 1 thereof. Compound A or a pharmaceutically acceptable salt thereof or a pharmaceutically acceptable solvate thereof, may be prepared using known methods, for example those disclosed in EP 0 306 228 and WO 94/05659. The disclosures of EP 0 306 228 and WO 94/05659 are incorporated herein by reference.

Compound A and pharmaceutically acceptable salts or solvates thereof have useful pharmaceutical properties. In particular, Compound A or a salt or solvate thereof is indicated to be useful for the treatment and/or prophylaxis of diabetes mellitus, conditions associated with diabetes mellitus and certain complications thereof; Alzheimer's Disease, mild cognitive impairment, psoriasis, asthma, atherosclerosis, metabolic syndrome, impaired glucose tolerance and impaired fasting glucose.

International Patent Application, Publication Number WO 00/28990 describes various modified release pharmaceutical compositions comprising insulin sensitisers, including Compound A and pharmaceutically acceptable salts or solvates thereof.

International Patent Application, Publication Number WO 00/28990 describes a method of treating Type 2 diabetes mellitus and conditions associated with diabetes mellitus, using certain pharmaceutical compositions, including modified release compositions, which provide a Threshold Plasma Concentration of Compound A or a pharmaceutically acceptable salt or solvate thereof.

International Patent Application Number PCT/EP2004/008843 (WO 05/013935) describes an oral dosage form comprising a first composition and a second composition, each composition comprising a pharmaceutically acceptable weak base, especially Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH.

Compound A is a pharmaceutically acceptable weak base.

Compound A and pharmaceutically acceptable salts or solvates thereof, in particular the maleate salt, have been found to exhibit marked pH dependent solubility, i.e. they are more soluble in the acidic conditions of the stomach (around pH 2) than in the near neutral conditions of the lower intestine (around pH 7).

It is an object of the present invention to provide an oral dosage form comprising Compound A or a pharmaceutically acceptable salt or solvate thereof, which provides a maximised beneficial effect, for example on glycaemic control, for an extended period of time. Such a dosage form is considered to be suitable for once daily administration. Such a dosage form is also indicated for administration in both fasted and fed states, with substantially no clinically relevant food effect.

The present invention is based on the finding that one or more objects of the invention can be accomplished by means of an oral dosage form in which Compound A or a pharmaceutically acceptable salt or solvate thereof is provided in pellet form in two different formulations which release drug at differing release rates on administration.

Accordingly, the present invention provides an oral dosage form comprising pellets of a first composition and pellets of a second composition, each composition comprising Compound A or a pharmaceutically acceptable salt or solvate thereof and a pharmaceutically acceptable carrier therefor, wherein the first and second compositions are arranged to release drug at differing release rates on administration, preferably such that the rate of release of the drug from the dosage form is substantially independent of pH.

The pellets are sized so that they may be loaded into capsule shells or compressed into tablets for oral dosage. Alternatively the pellets may be administered in granular form, typically being provided in unit dosage form in sachets or similar packaging.

Although the pellets may be formed into any suitable shape, in one embodiment the pellets are substantially spherical. Typically, the pellets have diameters in the range from about 0.25 to 2.5 mm, such as from about 0.3 to 2.0 mm, or if not spherical, are of such a size as to be capable of forming a substantially spherical pellet having such a diameter.

Suitably, the release rate of the drug from the first composition is substantially greater than from the second composition. It is envisaged that, the first composition is an immediate release composition. It is also envisaged that, the second composition is a modified release composition.

Alternatively, the rate of release of the first and second composition(s) from the dosage form is a modified release.

In one aspect, the first composition is arranged so that in use it releases substantially all of the Compound A or a pharmaceutically acceptable salt or solvate thereof, in the stomach.

In a further aspect, the second composition is arranged so that in use it releases substantially all of the Compound A or a pharmaceutically acceptable salt or solvate thereof in the small intestine.

Suitably, the dosage form is a capsule containing the first and second composition in pellet form.

In one aspect the oral dosage form is arranged to release the Compound A or a pharmaceutically acceptable salt or solvate thereof, such that the mean maximum plasma level concentration (“Cₘₐₓ”) value of the drug is maintained substantially independent of food during use, i.e. the observed Cₘₐₓ value is substantially similar in both fasted and fed states during use.

In another aspect the oral dosage form is arranged to release the Compound A or a pharmaceutically acceptable salt or solvate thereof, such that the mean area under the plasma concentration versus time curve over the dosing interval at steady state (“AUC”) is maintained substantially independent of food during use, i.e. the observed AUC is substantially similar in both fasted and fed states during use.

Thus, in a preferred aspect in operation the oral dosage form releases Compound A or a pharmaceutically acceptable salt or solvate thereof, so that both the Cₘₐₓ value and AUC observed on administration are maintained substantially independent of food during use, i.e. the observed Cₘₐₓ value and AUC are substantially similar in both fasted and fed states during use.

Suitably, the first composition is formulated so that it provides immediate release of Compound A or a pharmaceutically acceptable salt or solvate thereof, on contact with aqueous media. Suitably, the second composition is formulated so that it provides modified release of Compound A or a pharmaceutically acceptable salt or solvate thereof, on contact with aqueous media.

Suitably, the first composition comprises pellets that provide substantially immediate release of the drug, and the second composition comprises pellets that provide substantially immediate release of the drug provided with a modified release coating.

The compositions can be formed in any pellet-like form, such as granules, powders, spheroids or multiparticulates, especially granules, spheroids or multiparticulates, providing the required objective of the invention is met. In one embodiment the first and second compositions are in the form of granules. In another embodiment the first and second compositions are in the form of multiparticulates. In a further embodiment the first and second compositions are in the form of spheroids.

Most suitably, the dosage form is formulated so as to release drug to substantially the same extent in both the stomach and the intestines, i.e. is formulated to compensate for the pH dependency of Compound A.

The present invention also provides a process for preparing an oral dosage form comprising a first composition and a second composition, each composition comprising Compound A or a pharmaceutically acceptable salt or solvate thereof (‘the drug’) and a pharmaceutically acceptable carrier therefor, which process comprises at least the steps of sequentially or simultaneously:
(i) formulating the drug into the first composition; and
(ii) formulating the drug into the second composition;
and the steps of sequentially or simultaneously:
(iii) forming the first composition into a first mass of pellets;
(iv) forming the second composition into a second mass of pellets; and
(v) blending the first and second mass of pellets,
   to provide a dosage form in which the first and second mass of pellets release drug at differing release rates on administration, suitably so that the rate of release of the drug from the dosage form is substantially independent of pH.

In a preferred embodiment, the present invention provides a process for preparing an oral dosage form comprising a first composition and a second composition, each composition comprising Compound A or a pharmaceutically acceptable salt or solvate thereof (‘the drug’) and a pharmaceutically acceptable carrier therefor, which process comprises at least the steps of which process comprises at least the steps of sequentially or simultaneously:
(i) formulating the drug into the first composition; and
(ii) forming the first composition into a mass of pellets;
then
(iii) dividing the mass of pellets into a first mass and a second mass;
(iv) coating the second mass of pellets with a coating that provides modified release of the drug;
(v) blending the first mass and the coated second mass,
   to provide a dosage form in which the first and second mass of pellets release drug at differing release rates on administration, suitably such that the rate of release of the drug from the dosage form is substantially independent of pH.

Suitably the combined mass of pellets is loaded into capsule shells or sachets to form unit oral dosage forms, or compressed into tablets.

The pellets may be prepared using conventional excipients and formulation methods. Thus, the pellets typically comprise the active agent or agents along with excipients that impart satisfactory processing and compression characteristics such as diluents, binders and lubricants. Additional excipients may include disintegrants, flavourants, colorants, release modifying agents and/or solubilising agents such as surfactants, pH modifiers and complexation vehicles. Typically, the active agent and excipients are thoroughly mixed prior to pelleting or granulation.

As indicated above, the oral dosage form of the present invention is considered to be suitable for once daily administration and during use is indicated to provide a therapeutic effect over an extended period of time, such as up to 24 hours, for example, up to 12, 14, 16, 18, 20 and 24 hours, per unit dose.

As used herein, the term “modified release” means a composition which has been designed to produce a desired pharmacokinetic profile by choice of formulation. Modified release also includes modified release compositions in combination with non-modified release compositions. For example, the term “modified release” shall comprise delayed, pulsed and sustained release either alone or in any combination.

In one aspect the modified release composition provides delayed release of Compound A or a pharmaceutically acceptable salt or solvate thereof. Delayed release is conveniently obtained by use of a gastric resistant formulation such as an enteric formulation, for example immediate release pellets, such as multi-particulate spheres, are coated with a gastric resistant polymer. Suitable, gastric resistant polymers include polymers derived from methacrylates, cellulose acetate phthalate, polyvinyl acetate phthalate and hydroxypropyl methylcellulose phtahlate. Examples of such polymers include Eudragit L100-55™ (Poly(methacrylic acid, ethyl acrylate) 1:1) for example as Eudragit L30D-55™ or Eudragit FS 30D™, Aquateric™ (cellulose acetate phthalate), Sureteric™ (polyvinyl acetate phthalate), HPMCP-HP-55S™ (hydroxypropyl methylcellulose phtahlate).

The multiparticulates include drug-coated non-pareil substrates, such as lactose spheres, or drug-containing non-pareil substrates, such as drug-containing lactose spheres. Such multiparticulates are coated as required with an appropriate enteric formulation, for example a polymethacrylate polymer. An example of a suitable polymethacrylate polymer is Eudragit L100-55™ (Poly(methacrylic acid, ethyl acrylate) 1:1), for example as Eudragit L30D-55™ or Eudragit FS 30D™.

In a further aspect the modified release composition provides sustained release of Compound A or a pharmaceutically acceptable salt or solvate thereof, for example providing release of the active agent over a time period of up to 26 hours, up to 24 hours, up to 18 hours, or up to 16 hours; suitably in the range of 4 to 24 hours; preferably in the range of 12 to 24 hours.

Sustained release may be achieved by using immediate release pellets, such as multiparticulates, coated with semipermeable membranes. The multiparticulates include drug-coated non-pareil substrates, such as lactose spheres, or drug-containing substrates, such as drug-containing lactose/Avicel™ (microcrystalline cellulose) spheres. Such multiparticulates are coated as required with the appropriate semi-permeable membranes, such as ethylcellulose polymer.

In yet a further aspect the modified release composition provides pulsed release of Compound A or a pharmaceutically acceptable salt or solvate thereof, for example providing up to 4, for example 2, pulses of active agent per 24 hours.

Suitable materials for an immediate release composition, such as the first composition, include saccharoses, for example lactose and maltose. Most suitably, the immediate release composition is predominantly lactose. More suitably, the immediate release composition consists essentially of lactose and magnesium stearate.

A suitable dosage range for Compound A or a pharmaceutically acceptable salt or solvate thereof is up to 12 mg, for example, 1 to 12 mg. Thus, suitable dosage forms comprise 1, 2, 3, 4, 5, 6, 7, 8, 9, 10, 11 or 12 mg of Compound A or a pharmaceutically acceptable salt or solvate thereof.

Particular dosage forms comprise 2 to 4 mg of Compound A or a pharmaceutically acceptable salt or solvate thereof.

Particular dosage forms comprise 4 to 8 mg of Compound A or a pharmaceutically acceptable salt or solvate thereof.

Particular dosage forms comprise 8 to 12 mg of Compound A or a pharmaceutically acceptable salt or solvate thereof.

One dosage form comprises 1 mg of Compound A or a pharmaceutically acceptable salt or solvate thereof.

One dosage form comprises 2 mg of Compound A or a pharmaceutically acceptable salt or solvate thereof.

Preferred dosage forms comprise 4 mg of Compound A or a pharmaceutically acceptable salt or solvate thereof.

Preferred dosage forms comprise 8 mg of Compound A or a pharmaceutically acceptable salt or solvate thereof.

The amount of Compound A or a pharmaceutically acceptable salt or solvate thereof present in the first composition and the second composition may be varied in accordance with the desired dissolution profile.

For example, where the oral dosage form comprises 8 mg of Compound A or a pharmaceutically acceptable salt or solvate thereof, the dosage form suitably comprises one composition comprising 1 mg of Compound A or a pharmaceutically salt or solvate thereof, and another composition comprising 7 mg of Compound A or a pharmaceutically salt or solvate thereof. Alternatively, the tablet core may comprise one composition comprising 4 mg of Compound A or a pharmaceutically salt or solvate thereof, and another composition comprising 4 mg of Compound A or a pharmaceutically salt or solvate thereof. More suitably, the tablet core comprises one composition comprising 2 mg of Compound A or a pharmaceutically salt or solvate thereof, and another composition comprising 6 mg of Compound A or a pharmaceutically salt or solvate thereof. Preferably, the tablet core comprises a first composition as a substantially immediate release composition comprising 3 mg of Compound A or a pharmaceutically salt or solvate thereof, and a second composition as a modified release composition comprising 5 mg of Compound A or a pharmaceutically salt or solvate thereof.

Where the oral dosage form comprises 2 mg of Compound A or a pharmaceutically acceptable salt or solvate thereof, the tablet core suitably comprises a first composition comprising 0.75 mg of Compound A or a pharmaceutically salt or solvate thereof, and a second composition comprising 1.25 mg of Compound A or a pharmaceutically salt or solvate thereof.

Where the oral dosage form comprises 4 mg of Compound A or a pharmaceutically acceptable salt or solvate thereof, the tablet core suitably comprises a first composition comprising 1.5 mg of Compound A or a pharmaceutically salt or solvate thereof, and a second composition comprising 2.5 mg of Compound A or a pharmaceutically salt or solvate thereof.

By adjustment of the release rates of the first and second compositions, and adjusting other variables such as the surface area of the pellets, the release rates in the different environmental conditions can be harmonised to obtain comparable release rates under different body environments, and so achieve more constant dosing to a patient.

Dissolution rates may be assessed by in vitro testing in solutions of the appropriate pHs. For example, when comparing dissolution in the stomach and intestine, tests may be carried out initially at pH 1.5 with a transfer to pH 6.8 after 2 hours or 4 hours, as an assumed time for residence in the stomach before emptying into the intestines of a notional patient in respectively fasted and fed conditions.

As mentioned above, Compound A or a pharmaceutically acceptable salt or solvate thereof when administered in an oral dosage form of this invention is indicated to be useful for the treatment and/or prophylaxis of diabetes mellitus, conditions associated with diabetes mellitus and certain complications thereof; Alzheimer's Disease, mild cognitive impairment, psoriasis, asthma, atherosclerosis, metabolic syndrome, impaired glucose tolerance and impaired fasting glucose (hereinafter referred to as the ‘Disorders of the Invention’). Suitably, Compound A or a pharmaceutically acceptable salt or solvate thereof when administered in an oral dosage form of this invention is indicated to be useful in the treatment and/or prophylaxis of diabetes mellitus, conditions associated with diabetes mellitus and certain complications thereof. Suitably, Compound A or a pharmaceutically acceptable salt or solvate thereof when administered in an oral dosage form of this invention is indicated to be useful in the treatment and/or prophylaxis of Alzheimer's Disease. Suitably, Compound A or a pharmaceutically acceptable salt or solvate thereof when administered in an oral dosage form of this invention is indicated to be useful in the treatment and/or prophylaxis of mild cognitive impairment. Suitably, Compound A or a pharmaceutically acceptable salt or solvate thereof when administered in an oral dosage form of this invention is indicated to be useful in the treatment and/or prophylaxis of psoriasis. Suitably, Compound A or a pharmaceutically acceptable salt or solvate thereof is indicated to be useful in the treatment and/or prophylaxis of asthma. Suitably, Compound A or a pharmaceutically acceptable salt or solvate thereof when administered in an oral dosage form of this invention is indicated to be useful in the treatment and/or prophylaxis of atherosclerosis. Suitably, Compound A or a pharmaceutically acceptable salt or solvate thereof when administered in an oral dosage form of this invention is indicated to be useful in the treatment and/or prophylaxis of metabolic syndrome. Suitably, Compound A or a pharmaceutically acceptable salt or solvate thereof is indicated to be useful in the treatment and/or prophylaxis of impaired glucose tolerance. Suitably, Compound A or a pharmaceutically acceptable salt or solvate thereof when administered in an oral dosage form of this invention is indicated to be useful in the treatment and/or prophylaxis of impaired fasting glucose.

In a preferred embodiment the present invention provides a method for the treatment and/or prophylaxis of the Disorders of the Invention which method comprises administering an oral dosage form of this invention comprising Compound A or a pharmaceutically acceptable salt or solvate thereof, to a human or non-human mammal in need thereof.

In a further preferred embodiment the present invention provides an oral dosage form of the invention comprising Compound A or a pharmaceutically acceptable salt or solvate thereof for use in the treatment and/or prophylaxis of the Disorders of the Invention.

As used herein, the term “pharmaceutically acceptable” embraces compounds, compositions and ingredients for both human and veterinary use. For example the term “pharmaceutically acceptable salt” embraces a veterinarily acceptable salt. In particular, suitable pharmaceutically acceptable salted forms of Compound A include those described in European Patent Number 0 306 228 and International Patent Application, Publication Number WO 94/05659. A particularly preferred form of Compound A is the maleate salt.

Suitable pharmaceutically acceptable solvates include hydrates.

As used herein, the term “Cₘₐₓ” shall mean the mean maximum plasma level concentration.

As used herein the term “AUC” shall mean the mean area under the plasma concentration versus time curve over the dosing interval at steady state.

No adverse toxicological effects are indicated in the above mentioned treatments.

All publications, including but not limited to patents and patent applications, cited in this specification are herein incorporated by reference as if each individual publication were specifically and individually indicated to be incorporated by reference herein as though fully set forth.

The following Examples are intended to be by way of illustration rather than limitation of the present invention.

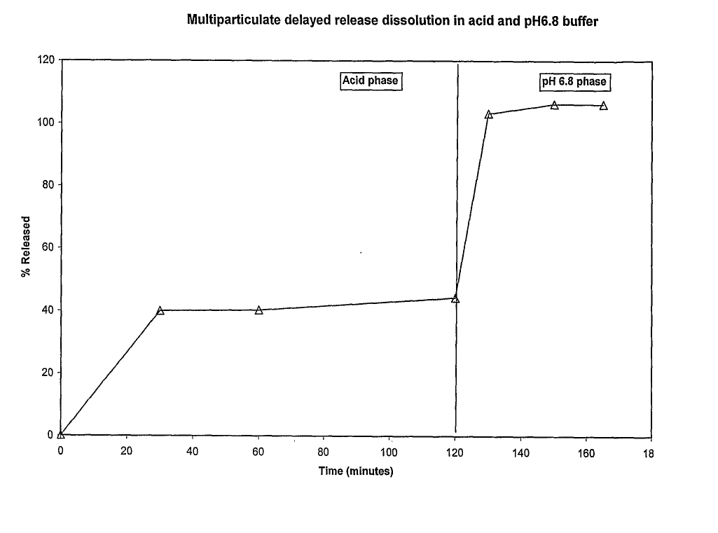

FIG. 1 is a graph of dissolution against time under different pH conditions for formulations of an oral dosage form in accordance with Example 1 below.

### EXAMPLE 1

Multiparticulates filled into a hard gelatine capsule shell to provide sustained release of Compound (A) and non-modified release (i.e. immediate) release of Compound (A).

The drug layered multiparticulates are prepared by fluid bed coating sugar spheres with the required amount of Compound (A). The drug layered multiparticulates are then seal coated with Opadry clear. At this stage the product is used as the non-modified (i.e. immediate) release capsule component.

For the sustained release dose, a portion of the non-modified (i.e. immediate) release multiparticulates are coated with an enteric coat and then a final seal coat.

| | |
|---|---|
| Non-modified (i.e. Immediate) Release | |
| Component A | mg/tablet |

1) Drug layered multiparticulates

| | |
|---|---|
| Compound (A) | 3 mg (pfb) |
| Polysorbate 80 | 0.735 |
| Opadry Clear | 2.209 |
| Sugar Spheres to | 80.535 |

2) Seal Coat

| | |
|---|---|
| Opadry Clear | 1.215 |
| Non-Modified Release Component Weight | 81.750 |

| | |
|---|---|
| Modified component A | mg/tablet |

1) Enteric Coat

| | |
|---|---|
| Immediate Release Component A | 136.25* |
| Methacrylic Acid Copolymer | 34.688 |
| Talc | 8.000 |
| Triethyl Citrate | 5.188 |

2) Seal Coat

| | |
|---|---|
| Opadry Clear | 3.750 |
| Modified Release Component Weight | 187.876 |

Equivalent to 5 mg (pfb)

### EXAMPLE 2

For preparation of a capsule containing multiple pellet cores, firstly pellets are formed using the following mixture:

| | mg/capsule |
|---|---|
| Compound (A) | 8 (pfb) |
| Microcrystalline cellulose | 133.5 |
| Lactose monohydrate | to 267 |

A portion of the pellets equivalent to 3 mg/capsule of Compound (A) is set aside as the immediate release component of the capsule.

The remaining portion of the pellets, equivalent to 5 mg/capsule of Compound (A), is coated with Eudragit L100-55, a gastric resistant polymer to provide a delayed release component.

The enteric coat consists of:

| | % w/w |
|---|---|
| Eudragit L30 D-55 (30% aqueous dispersion) | 76.8 |
| Triethyl Citrate | 7.7 |
| Talc Alphafil 500 | 15.5 |

The two components are blended and loaded into capsule shells.

### EXAMPLE 3

For preparation of a capsule containing immediate release and delayed release components in multiparticulate form, multiparticulates are prepared by forming drug layered lactose spheres (the drug layer being 8 mg of Compound (A) as pure free base (pfb) per dose), and the coating with either Eudragit L30D-55 or EudragitFS 30D, pH dependent polymers.

Drug-layered multiparticulates are prepared by fluid-bed coating of lactose spheres with Compound (A). The drug-layered multiparticulates therefore consist of:

| | % w/w |
|---|---|
| Compound (A)* | 5.40 |
| Opadry Clear | 3.0 |
| Polysorbate 80 (Tween 80) | 1.0 |
| Purified Water | q.s. |
| Lactose spheres (25-30 mesh) | 200 mg |

*This is based on Purity (as is) 99.2% w/w. Pure Free Base: 74.9% w/w.

The drug layered multiparticulates are then seal-coated with 2%, by weight, of film former Opadry Clear.

A portion of the pellets equivalent to 3 mg/capsule of Compound (A) is set aside as the immediate release component of the capsule.

The remaining portion of the pellets, equivalent to 5 mg/capsule of Compound (A), is coated with a gastric resistant polymer to provide a delayed release component.

The enteric coat consists of:

| | % w/w |
|---|---|
| Eudragit L30 D-55 (30% aqueous dispersion) | 10-25 |
| Triethyl Citrate | 15* |
| Talc Alphafil 500 | 23.0* |
| Purified Water** | q.s. |

*These percentages are based on the solid content of the Eudragit
**Sufficient water is added such that the total solids content is 16%.

| | |
|---|---|
| Eudragit FS 30D (30% aqueous dispersion) | 10-15 |
| Glyceryl Monostearate, NF | 3.0* |
| Triethyl Citrate | 1.0* |
| Purified Water** | q.s. |

*These percentages are based on the solid content of the Eudragit
**Sufficient water is added such that the total solids content is 16%.

The multiparticulates can be admixed and filled into capsules.

### EXAMPLE 4

Sustained release multiparticulates are prepared by coating drug layered lactose spheres (the drug layer being 8 mg of Compound (A) as pure free base (pfb) per dose) with ethylcellulose polymer (Surelease).

The drug layered multiparticulates consist of:

| | % w/w |
|---|---|
| Compound (A)* | 5.40 |
| Opadry Clear | 3.0 |
| Polysorbate 80 (Tween 80) | 1.0 |
| Purfied Water | q.s. |
| Lactose spheres (25-30 mesh) | 200 mg |

*This is based on Purity (as is) 99.2% w/w. Pure Free Base: 74.9% w/w.

The drug layered multiparticulates are seal coated with 2%, by weight, with film former Opadry Clear.

A portion of the pellets equivalent to 3 mg/capsule of Compound (A) is set aside as the immediate release component of the capsule.

The remaining portion of the pellets, equivalent to 5 mg/capsule of Compound (A), is coated with a semipermeable membrane polymer to provide a sustained release component.

The semipermeable membrane consists of:

| | % w/w |
|---|---|
| Surelease Clear | 10-20 |
| Purified Water* | q.s. |

*Sufficient water is added such that the total solids content is 15%.

The multiparticulates can be admixed and filled into capsules or compressed into tablets to provide the desired release profile.

1-19. (canceled)

20. An oral dosage form comprising pellets of a first composition and pellets of a second composition, each composition comprising a drug, wherein the drug is 5-[4-[2-(N-methyl-N-(2 pyridyl)amino)ethoxy]benzyl]thiazolidine-2,4-dione, or a pharmaceutically acceptable salt or hydrate thereof, and a pharmaceutically acceptable carrier therefor, wherein the first and second compositions are arranged to release drug at differing release rates on administration.

21. An oral dosage form according to claim 20, which is arranged such that the rate of release of the drug from the dosage form is substantially independent of pH.

22. An oral dosage form according to claim 20, wherein the release rate of the drug from the first composition is substantially greater than from the second composition.

23. An oral dosage form according to claim 20, wherein the first composition is an immediate release composition.

24. An oral dosage form according to claim 20, wherein the second composition is a modified release composition.

25. An oral dosage form according to claim 24, wherein the modified release composition is a delayed release, sustained release or pulsed release composition.

26. An oral dosage form according to claim 20, wherein the first composition is arranged so that in use it releases substantially all of the drug in the stomach.

27. An oral dosage form according to claim 20, wherein the second composition is arranged so that in use it releases substantially all of the drug in the small intestine.

28. An oral dosage form according to claim 20, which dosage form is arranged to release the drug such that the mean maximum plasma level concentration value of the drug is maintained substantially independent of food during use.

29. An oral dosage form according to claim 20, which dosage form is arranged to release the drug such that the mean area under the plasma concentration versus time curve over the dosing interval at steady state is maintained substantially independent of food during use.

30. An oral dosage form according to claim 20, which dosage form is arranged to release the drug so that both the mean maximum plasma level concentration value and the mean area under the plasma concentration versus time curve over the dosing interval at steady state observed on administration are maintained substantially independent of food during use.

31. An oral dosage form according to claim 20, wherein the first composition is formulated so that it provides immediate release of the drug on contact with aqueous media.

32. An oral dosage form according to claim 20, wherein the second composition is formulated so that it provides modified release of the drug on contact with aqueous media.

33. An oral dosage form according to claim 20, wherein the dosage form is a tablet form.

34. An oral dosage form according to claim 20, wherein the dosage form is a capsule.

35. A process for preparing an oral dosage form comprising a first composition and a second composition, each composition comprising a drug, wherein the drug is 5-[4-[2-(N-methyl-N-(2 pyridyl)amino)ethoxy]benzyl]thiazolidine-2,4-dione, or a pharmaceutically acceptable salt or hydrate thereof, and a pharmaceutically acceptable carrier therefor, according to claim 20, which process comprises at least the steps of sequentially or simultaneously:
formulating the drug into the first composition; and
formulating the drug into the second composition;
and the steps of sequentially or simultaneously:
forming the first composition into a first mass of pellets;
forming the second composition into a second mass of pellets; and
blending the first and second mass of pellets,
to form a dosage form in which the first and second mass of pellets release the drug at differing release rates on administration, such that the rate of release of the drug from the dosage form is substantially independent of pH.

36. A process for preparing an oral dosage form comprising a first composition and a second composition, each composition comprising a drug, wherein the drug is 5-[4-[2-(N-methyl-N-(2 pyridyl)amino)ethoxy]benzyl]thiazolidine-2,4-dione, or a pharmaceutically acceptable salt or hydrate thereof, and a pharmaceutically acceptable carrier therefor, according to claim 20, which process comprises at least the steps of sequentially or simultaneously:
(i) formulating the drug into the first composition; and
(ii) forming the first composition into a mass of pellets; then
(iii) dividing the mass of pellets into a first mass and a second mass;
(iv) coating the second mass of pellets with a coating that provides modified release of the drug;
(v) blending the first mass and the coated second mass,
to form a dosage form in which the first and second mass of pellets release the drug at differing release rates on administration, such that the rate of release of the drug from the dosage form is substantially independent of pH.

37. A process according to claim 35, wherein the blended mass of pellets is loaded into capsule shells to form unit oral dosage forms

38. A method for the treatment or prophylaxis of a disorder selected from diabetes mellitus, conditions associated with diabetes mellitus, Alzheimer's Disease, mild cognitive impairment, psoriasis, asthma, atherosclerosis, metabolic syndrome, impaired glucose tolerance and impaired fasting glucose, in a human or non-human mammal, which method comprises administering an oral dosage form according to claim 20, to a human or non-human mammal in need thereof.
